(19) United States
(12) Patent Application Publication
(10) Pub. No.: US 2023/0233874 A1
(43) Pub. Date: Jul. 27, 2023

(54) A PHOTOBIOMODULATION THERAPY LOW-LEVEL LASER TARGETING SYSTEM

(71) Applicant: COSMETIC EDGE PTY LTD, Kew (AU)

(72) Inventor: Masoud JAFARZADEH, Kew (AU)

(21) Appl. No.: 18/000,054

(22) PCT Filed: Jun. 4, 2021
(86) PCT No.: PCT/AU2021/050558

§ 371 Date: Nov. 28, 2022

(30) Foreign Application Priority Data
Jun. 4, 2020 (AU) 2020901835

(51) Int. Cl. A61N 5/067; A61B 5/01

(52) U.S. Cl. CPC A61N 5/067; A61B 5/01

(57) ABSTRACT

A photobiomodulation therapy low-level laser targeting system has a controller, a low lever laser emitter controlled by the controller and a projector operably coupled to the emitter and controlled by the controller to control the projection direction of light from the emitter. The controller has a targeting controller configured for controlling the projector to project light from the emitter onto a skin surface target area in use to target a subdermal target region.

### FIELD OF THE INVENTION

This invention relates generally to photobiomodulation. More particularly, this invention relates to a photobiomodulation therapy low-level laser targeting system.

### BACKGROUND OF THE INVENTION

Photobiomodulation therapy uses a low energy level lasers to apply red and near infra-red light to injuries or lesions to improve wound and soft tissue healing, reduce inflammation and give relief for both acute and chronic pain by non-thermal photochemical effect. The light triggers biochemical changes within cells wherein photons are absorbed by cellular photoreceptors to trigger chemical changes.

The present invention seeks to provide a low-level laser targeting system for photobiomodulation therapy, which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE DISCLOSURE

There is provided herein a photobiomodulation laser targeting system which uses a low-level laser to treat a variety of internal tissue injuries, trauma, ulcers, inflammation and the like and/or control infection.

The system comprises a controller and a low-level laser emitter coupled to the controller. The emitter may emit red and near infrared light typically in the range of 660 nm - 905 nm at low power of between 10 mW - 500 mW to deliver a power density (irradiance) of approximately 5 W/cm² on the skin surface target area.

The system further comprises a projector operably coupled to the emitter and controlled by the controller to control projection direction of light from the emitter, such as in two axes.

The controller comprises a targeting controller configured for controlling the projector to direct light from the emitter onto a skin surface target area in use to target a subdermal target region.

The system may be precisely targeted subdermal target region using geospatial data which may be obtained from medical scanning devices and procedures such as CT scanners, CAT-scanners, MRI scanners, colonoscopies, endoscopies, x-rays, mammograms, ultrasound investigations and the like. The system may comprise a computer aided geospatial editor to allow a physician to configure geospatial data for targeting the subdermal target region with respect to a 3D patient body representation.

The system may further comprise a ranging controller operably coupled to a sensor for determining a target region and wherein the targeting controller controls the projector according to the target region determined by the ranging controller.

The sensor may comprise a thermal sensor configured for determining a skin surface heat map topography indicative of inflammation or the like and wherein the targeting controller specifically targets areas of elevated temperature.

The sensor may comprise a vision sensor configured for identifying an applied skin marking, such as a visible or infrared visible point boundary. As such, a physician may mark the treatment area which is detected by the vision sensor for controlling the targeting of the targeting controller.

The ranging controller may further use image processing to process image data obtained from a camera device to identify various regions of the body for targeting, such as by way of shape and/or object recognition. As such, a physician may specify that the right knee is to be targeted for treatment and wherein the ranging controller identifies the location of the right knee using image processing.

The ranging controller may further adjust the targeting of the targeting controller if the position of the projector moves with respect to the subdermal target region.

In embodiments, the patient usable form of the system comprises a small form applicator device having the emitter and projector therein which is operably coupled to a user interface device, such as a smart phone, tablet computing device or the like. The user interface device may execute a software application thereon for control, including setting of settings, marking of target regions and the like. The user interface device may display a treatment region augmented with image data obtained from a camera thereof. The applicator device may use from sensors of the user interface device, such as image and/or gyroscopic sensors thereof.

In embodiments, the applicator device is a small form factor device which may attach to the user interface device and may have a rechargeable battery therein to power the emitter or alternatively draw power from the user interface device. As such, for home-based photobiomodulation therapy, the user may hold the user interface device and attached applicator device in one hand wherein the system uses the ranging controller (by thermal sensing, or vision sensing to detect a marking or recognise a body portion) to precisely control the targeting of the targeting controller irrespective of the relative positioning of the projector from the subdermal target region.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a photobiomodulation laser targeting system in accordance with an embodiment;

FIG. 2 shows exemplary apparatus of the system in accordance with an embodiment;

FIG. 3 shows an exemplary user interface;

FIG. 4 shows a side elevation view of an applicator of the apparatus of FIG. 2; and

FIG. 5 shows a front elevation view of the applicator of the apparatus of FIG. 2.

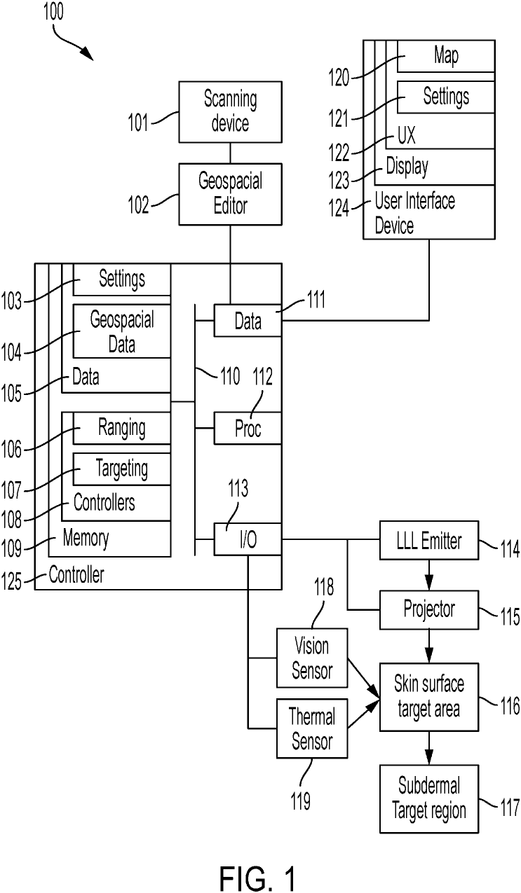

### DESCRIPTION OF EMBODIMENTS

A photobiomodulation laser targeting system 100 comprises a controller 125 and a low-level laser emitter 114 controlled by the controller 125 via an l/O interface 113.

The system 100 further comprises a projector 115 operably coupled to the emitter 114 and controlled by the controller 125.

The controller 125 comprises a processor 112 for processing digital data. In operable communication with the processor 112 across a system bus 110 is a memory device 109 configured for storing digital data including computer program code instructions. The computer program code instructions may be logically divided into various computer program code controllers 108 and associated data 105. In use, the processor 112 fetches these computer program code instructions and associated data from the memory device 109 for interpretation and execution for implementing the control functionality described herein.

The controller 125 comprises a targeting controller 107 configured for controlling the projector 115 to direct light from the emitter 114 onto a skin surface target area 116 to target a subdermal target region 117.

The emitter 114 may emit red and near infrared light in the range of 660 nm -905 nm at low power of between 10 mW - 500 mW to deliver a power density (irradiance) of up to approximately 5 W/cm² on the skin surface target area 116.

The projector 115 may direct the light in two axes, thereby allowing the system 100 to direct light onto skin surface target areas 116 of differing shapes and sizes.

The projector 115 may comprise a mechanical gimbal which controls the orientation of the emitter 114. In alternative embodiments, a mechanical gimbal may adjust a mirror or prism against or through which the light is reflected or propagated.

In embodiments, the projector 115 comprises at least one rotating prism and wherein the emitter 114 is operated at specific rotational offsets of the at least one rotating prism to target the skin surface target area 116.

In embodiments, the projector 115 may comprise a beamforming lens. The beamforming lens may form a pinpoint for XY raster scanning or alternatively a line which is swept across the skin surface targeted treatment area 116.

The controller 125 may be configured with geospatial data 104 representing the subdermal target region 117.

The controller 125 may comprise a data interface 111 for receiving geospatial data 104 from a medical scanner device 101 or procedure. The medical scanning device 101 or procedures comprising CT scanner, CAT-scanner, MRI scanner, colonoscopy, endoscopy, x-ray scanner, mammogram, ultrasound investigation and the like.

The system 100 may comprise a computer aided modelling geospatial editor 102 for configuring geospatial data received from the patient scanner 101. In embodiments, the geospatial editor 102 may comprise a 3D model representation of a patient body which may be customised according to patient specific parameters.

With reference to data received from the patient scanner 101, a physician may configure the geospatial data 104 representing the subdermal target region 117 within the 3D model. For example, with reference to frontal and lateral x-ray data, the physician may configure the geospatial data 104 to represent the appropriate the subdermal target region 117.

As such, in use, the targeting controller 117 targets the subdermal targeting region 117 specified by the geospatial data 104.

The targeting controller 117 may target the subdermal target region 117 with the geospatial data 104 with reference to relative positioning of the projector 115 to the subdermal target region 117.

In one embodiment, the projector 115 may be placed at a set position with respect to the patient and wherein the targeting controller 117 targets the skin surface target area 116 and therefore the subdermal target region 117 thereunderneath with respect to the relative position of the projector 115 and the patient. In further embodiments, the targeting controller 117 may be configured with positional offsets, such as X, Y and Z coordinates representing the relative positioning of the projector 115 from the patient.

In embodiments, the controller 125 comprises a ranging controller 106 operably coupled to a sensor for determining a target region (such as the skin surface target area 116 or subdermal target region 117) and wherein the targeting controller 107 controls the projector 115 according to the target region and determined by the ranging controller 106.

In embodiments, the sensor comprises a thermal sensor 119 configured for determining skin surface heat map topography. The thermal sensor 119 may comprise an infrared camera orientated towards the skin of the patient. Alternatively, the thermal sensor 119 may comprise an infrared temperature sensor which emits an infrared energy beam focused by a lens to a surface of the skin surface target area 116 to determine the temperature thereof according to the energy of the reflected beam.

The ranging controller 106 may determine a region of elevated temperature for targeting by the targeting controller 107. A region of elevated temperature may be indicative of inflammation requiring treatment.

In further embodiments, the sensor comprises a vision sensor 118. In one embodiment, the vision sensor 118 is configured for identifying a skin marking. For example, a physician may mark a treatment area using a skin marking either using visible or infrared visible dye which is detected by the vision sensor 118. The skin marking may comprise a point and wherein the targeting controller 107 targets a region surrounding the point. In alternative embodiments, the skin marking may comprise a boundary and wherein the targeting controller 107 targets a region within the boundary. The targeting controller 107 may employ boundary area analysis image processing on image data obtained by the vision sensor to determine the area within a marked boundary for targeting.

In embodiments, when making the skin marking, the physician may indicate the skin marking with reference to image data captured by the vision sensor 118 displayed by a digital display 123 the system 100, thereby allowing the ranging controller 106 to thereafter target the indicated marking. For example, once having made a marking, the physician may tap the digital display 123 to indicate marking. Similarly, the physician may tap the display 123 within a marked boundary, thereby allowing the range controller 106 to subsequently target the area determined within the boundary.

In further embodiments, the sensor comprises a camera and wherein the ranging controller 106 uses shape detection and/or object recognition to determine regions of a body for targeting. For example, the ranging controller 106 may recognise a portion of the patient’s body using shape and/or object recognition for targeting by the targeting controller 107. Example, for targeting a knee, the ranging controller 106 may determine the boundary of the leg using shape detection and furthermore determine the location of the knee between the upper leg and the lower leg using shape for object recognition.

In embodiments, using the aforedescribed 3D model, the user may select a portion of the patient’s body for treatment. For example, the 3D model may be displayed on the display 123 when the physician may select the knee from the displayed 3D model. As such, the range controller 106 may use the shape and/or object recognition to recognise the knee selected from the 3D model for targeting.

The ranging controller 106 and targeting controller 107 may adjust targeting in real-time including if the position of the skin surface targeted treatment area 116 moves with respect to the projector 115 in use.

The controller 125 may be configured with adjustable settings 103 which, in embodiments may, for example, be used to adjust the treatment program. In embodiments, the settings 103 be used to control the emitter 114 and the projector 115, including for setting whether constant or pulsed light is applied, the light energy level, the dosage level, the treatment time period and treatment frequency.

The emitter 114 and the projector 115 may be controlled by the settings 103 to adjust the penetration depth. Penetration depth may be controlled by the energy level of the emitter 114.

In alternative embodiments, penetration depth may be controlled geometrically with respect to the relative positioning of the projector 115 and the subdermal target region 117. For example, as the position of the projector 115 moves with respect to the patient, the incident point on the skin surface target area 116 may be controlled by the targeting controller 107 to target the same depth of the subdermal target region 117 irrespectively.

In embodiments, the controller 115 is in operable communication with a user interface device 124. The user interface device 124 may take the form of a mobile communication device, tablet computing device or the like. The user interface device 124 may execute a software application thereon.

The user interface device 124 may comprise the digital display 123 configured for displaying a user interface 122 for controlling the operation of the controller 125.

The user interface 122 may display operational parameters. The user interface 122 may display settings 121 which may be adjusted.

The user interface 122 may display an augmented vision map representation 120 of the skin surface target area 116, augmented with image data obtained from a camera of the user interface device 124. In embodiments, the map representation 120 is interactive for marking the treatment boundary for targeting by the targeting controller 107.

In embodiments, a small form factor handheld applicator device 127 comprises the emitter 114 and projector 115. The applicator device 127 may be operably coupled to the user interface device 124. The applicator device 127 may comprise a rechargeable battery therein for powering the emitter 114 or may draw power from the user interface device 124. The applicator device 127 may be physically attached to the user interface device 124 or separated therefrom.

In accordance with this embodiment, the projector 115 may control the laser beam depending on the orientation and position of the user interface device 124 with respect to the subdermal target region 117.

For example, where the applicator device 127 is physically attached to the user interface device 124, both can be held in one hand during home-based photobiomodulation therapy wherein the ranging controller 106 works in conjunction with the vision sensor 118 or thermal sensor 119 to adjust the targeting of the targeting controller 107. In embodiments, the controller uses image data obtained from an image sensor of the user interface device 124 for targeting, thereby avoiding image sensing componentry and associated computation requirements of the applicator device 127 itself.

In embodiments, the applicator device 127 comprises gyroscopic sensors to determine the orientation of the applicator device and wherein the projector 115 further controls the laser beam depending on the orientation of the applicator device 127 determined by the gyroscopic sensors. Similarly, the system 100 may use gyroscopic sensors of the user interface device 124, thereby avoiding the applicator device 127 requiring separate gyroscopic sensors.

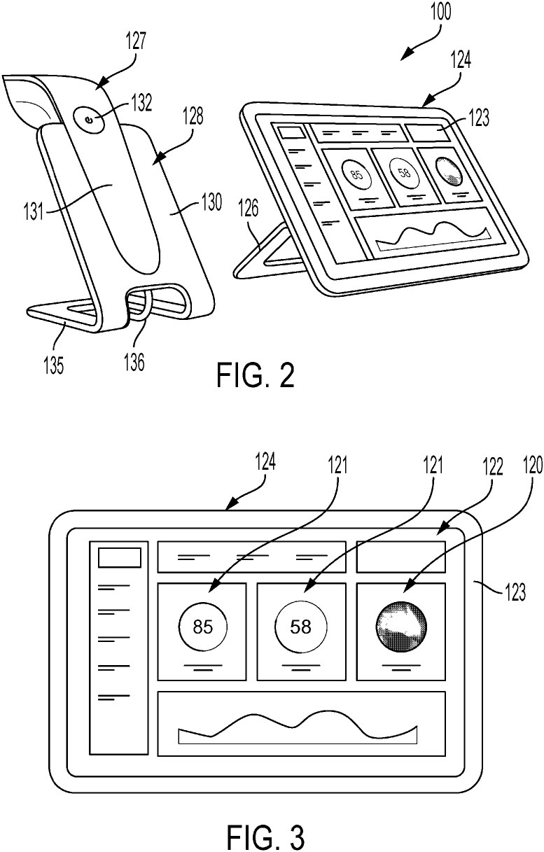

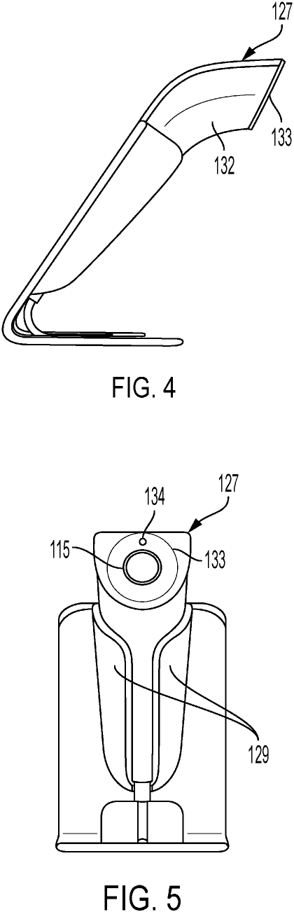

FIGS. 2 - 5 show an exemplary physical apparatus of the system 100 suited for desktop application. FIG. 2 shows the controller 125 taking the form of a tablet computing device having the digital display 123 and a supportive kickstand 126 therebehind.

FIG. 3 shows an exemplary graphical user interface 122 displayed by the digital display 123 which may comprise settings controls 121, the map representation 120 and other graphical user interface elements.

The apparatus may comprise a separate applicator 127 having the LLL emitter 114 and projector 115 therein.

The applicator 127 may be held within an applicator cradle 128 which may comprise a stand plate 130 and a footplate 135. With reference to FIG. 5, the stand plate 130 may comprise flanges 129 holding rear sides of the applicator 127 recessed behind a stand plate 130 of the cradle 128. The applicator 125 may comprise a handle stem 131 and a projection head 132. The applicator 125 may comprise a hardwired control cable 136 from a distal end of the handle stem 131. The applicator 127 may comprise a control button 132 for controlling the operation of the applicator 127.

The projection head 132 may comprise a face 133 having the projector 115 having adjustable optics located centrally therein and from which the light is projected onto the skin surface target area 116. The face 133 may further comprise an infrared camera 134 as the vision sensor 118.

In embodiments, the applicator 127 may remain within the cradle 128 during photobiomodulation therapy. In alternative embodiments, the applicator 127 is handheld during photobiomodulation therapy wherein targeting thereof is controlled by the ranging controller and/or gyroscopic sensors thereof.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

The term “approximately” or similar as used herein should be construed as being within 10% of the value stated unless otherwise indicated.

1. A photobiomodulation therapy low-level laser targeting system comprising:
a controller;
a low lever laser emitter controlled by the controller; and
a projector operably coupled to the emitter and controlled by the controller to control the projection direction of light from the emitter, wherein:
the controller comprises a targeting controller configured for controlling the projector to project light from the emitter onto a skin surface target area in use to target a subdermal target region, and
the controller is configured with geospatial data representing the subdermal target region and wherein the targeting controller is configured for controlling the projector depending on relative positioning of the projector with respect to the skin surface target area and the geospatial data.

2. The system as claimed in claim 1, wherein the projector directs the light in two axes.

3. The system as claimed in claim 2, wherein the projector comprises a mechanical gimbal which controls the orientation of the emitter.

4. The system as claimed in claim 2, wherein the projector comprises a mechanical gimbal which adjusts a mirror or prism against or through which the light is reflected or propagated.

5. The system as claimed in claim 2, wherein the projector comprises at least one rotating prism and wherein the emitter is operated at specific rotational offsets of the at least one rotating prism to target the skin surface target area.

6. The system as claimed in claim 2, wherein the projector comprises a beamforming lens.

7. The system as claimed in claim 6, wherein the beamforming lens may form a pinpoint for XY raster scanning.

8. The system as claimed in claim 6, wherein the beamforming lens forms a line which is swept across the skin surface targeted treatment area.

9. The system as claimed in claim 1, wherein the projector is set at a preconfigured position with respect to the subdermal target region.

10. The system as claimed in claim 10, wherein the controller is configured with relative positional coordinates representing a relative position of the projector with respect to the subdermal target region.

11. The system as claimed in claim 1, wherein the controller comprises a data interface for receiving geospatial data obtained from at least one of a medical scanning devices and procedures comprising at least one of a CT scanner, CAT-scanner, MRI scanner, colonoscopy, endoscopy, x-ray scanner, mammogram and ultrasound investigation.

12. The system as claimed in claim 1, further comprising a computer aided modelling geospatial editor for editing the geospatial data with reference to a 3D patient model.

13. The system as claimed in claim 1, wherein an incident point on the skin surface target area is controlled according to a penetration depth depending on relative positioning of the projector and the subdermal target region.

14. The system as claimed in claim 1, further comprising a ranging controller operably coupled to a sensor for determining a target region and wherein the targeting controller controls the projector according to the target region determined by the ranging controller.

15. The system as claimed in claim 14, wherein the sensor comprises a thermal sensor configured for determining a skin surface heat map topography.

16. The system as claimed in claim 15, wherein the targeting controller is configured for targeting areas of the surface heat map topography exceeding a temperature threshold.

17. The system as claimed in claim 15, wherein the thermal sensor comprises an infrared camera.

18. The system as claimed in claim 15, wherein the thermal sensor comprises an infrared temperature sensor which emits an infrared energy beam focused by a lens to a surface of the skin surface target area.

19. The system as claimed in claim 14, wherein the sensor comprises a vision sensor configured for identifying a skin marking.

20. The system as claimed in claim 19, wherein the skin marking is a point and wherein the targeting controller is configured for targeting a region around the point.

21. The system as claimed in claim 19, wherein the skin marking is a marked boundary and wherein the targeting controller is configured for targeting a region within the boundary.

22. The system as claimed in claim 21, wherein the targeting controller employs boundary area analysis image processing on image data obtained by the vision sensor to determine the area within a marked boundary for targeting.

23. The system as claimed in claim 19, wherein the skin marking is a visible skin marking.

24. The system as claimed in claim 19, wherein the skin marking is an infrared visible skin marking.

25. The system as claimed in claim 19, wherein skin marking is indicated with reference to a display of image data captured by the vision sensor and wherein the ranging controller is configured to thereafter target the indicated marking.

26. The system as claimed in claim 14, wherein the sensor is a camera and wherein the ranging controller uses image processing on image data received therefrom to determine the target region.

27. The system as claimed in claim 26, wherein the ranging controller targets a selected portion of a 3D patient model.

28. The system as claimed in claim 27, wherein the ranging controller uses image recognition to recognise the selected portion.

29. The system as claimed in claim 1, wherein the system comprises a small form applicator device comprising the emitter and projector therein and wherein the applicator device is operably coupled to a user interface device having a digital display and wherein the digital display displays a user interface for controlling the controller thereon.

30. The system as claimed in claim 29, wherein the applicator device attaches to the user interface device and wherein the controller further comprises a ranging controller operably coupled to a sensor for determining a target region and wherein the targeting controller controls the projector according to the target region determined by the ranging controller irrespective of the relative orientation and position of the user interface device and the transdermal target region.

31. The system as claimed in claim 30, wherein the sensor is a camera of the user interface device.

32. The system as claimed in claim 29, wherein the user interface displays a treatment area augmented with image data obtained from a camera of the user interface device.

33. The system as claimed in claim 29, wherein the applicator device is physically separate from the user interface device and the applicator device comprises gyroscopic sensors to determine the orientation of the applicator device and wherein the projector controls the laser beam depending on the orientation of the electronic device determined by the gyroscopic sensors.

34. The system as claimed in claim 29, wherein the applicator device is physically attached to the user interface device and the projector controls the laser beam depending on the orientation of the electronic device determined by gyroscopic sensors of the user interface device.

35. The system as claimed in claim 29, wherein the user interface device displays an augmented vision map representation of the skin surface target area augmented with image data obtained from a camera of the system.

36. The system as claimed in claim 35, wherein the map representation is interactive for marking the treatment boundary for targeting by the targeting controller.
